(19) United States
(12) Patent Application Publication
(10) Pub. No.: US 2004/0115173 A1
(43) Pub. Date: Jun. 17, 2004

(54) Method of treating inflammation, particularly diabetes

(76) Inventors: Daniela Santoli, Bryn Mawr, PA (US); Clara Brando, Bethesda, MD (US)

Correspondence Address: HOWSON AND HOWSON, ONE SPRING HOUSE CORPORATION CENTER, BOX 457, 321 NORRISTOWN ROAD, SPRING HOUSE, PA, 19477, US

(21) Appl. No.: 10/467,968

(22) PCT Filed: Feb. 15, 2002
(86) PCT No.: PCT/US02/03383

(51) Int. Cl. A61K 45/00

(52) U.S. Cl. 424/93.7; 424/85.2

(57) ABSTRACT

A method of treating inflammation and/or diabetes in a mammal involves administrering to the mammal an effective anti-inflammatory amount of allogeneic lymphocytic T cells. Among useful cells are TALL-104 cells, ATCC Accession No. CRL 11386, which cells have been modified by stimulation in vitro by treatment with a cytokine and gamma irradiation at a dose suitable to irreversibly arrest cell proliferation. These modified cells are characterized by irreversibly arrested cell proliferation and non-MHC restricted cytotoxic activity. Among other useful cells are TALL-106 cells.

### STATEMENT REGARDING FEDERALLY SPONSORED RESEARCH

This application was supported by National Institute of Health Grant Nos. CA 20833-25 and CA 10815-30. The United States government has certain rights in this invention.

### BACKGROUND OF THE INVENTION

A variety of diseases and disorders in mammals, particularly humans, are related to uncontrolled or inappropriate chronic inflammatory responses in the mammals. Such uncontrolled responses lead to a variety of autoimmune disorders. For example, in human type I diabetes, auto-reactive lymphocytes infiltrate the pancreatic islets, progressively destroying the β-cells of the pancreatic islets. The end result is that insulin is no longer produced, and a chronic disorder occurs. For many disorders, like diabetes, the current treatments, e.g., the exogenous administration of insulin, are not optimal for the health of the mammal.

Ongoing studies have shown that, in addition to autoreactive (antigen specific) lymphocytes, other cell types are known to control autoimmunity (Shevach, E. M. 2000 Annu. Rev. Immunol. 18:423). Both lymphokines and chemokines are also involved in controlling autoimmunity, including type I diabetes (Lloyd, C. et al. 1998 Curr. Opin. Nephrol. Hypertens. 7:281; Grewal, I. S. et al. 1997 J. Immunol. 159:401; and Youssef, S. et al. 1999 J. Autoimmmun. 13:21).

Specifically, CD4+ Th.1 cells have proven to be pro-diabetic in the Non-Obese diabetic (NOD) mouse model by virtue of IL-2 and IFN-γ production, whereas Th.2 type lymphokines, such as IL-4 and IL-10, have anti-inflammatory properties, and have been shown to have an anti-diabetogenic effect in NOD mice (Hancock, W. W., et al. 1995 Am. J. Pathol 147:1193). A recent report has indicated that natural killer T cells (i.e., NK cells, a double-negative T cell subset which expresses the Vα24 chain of the TCR) have a role in ensuring immunological tolerance, including prevention of diabetes in NOD mice (Hammond, K. J. L., et al. 1998 J. Exp. Med. 187:1047). While the authors correlated this anti-diabetogenic effect with IL-4 production, another report suggests that NK T cells have a direct cytostatic effect on autoreactive lymphocytes which does not require IL-4 production (Falcone, M., et al. 1999 J. Exp. Med. 190: 963). An anti-diabetogenic effect has also been observed following administration of IL-1 (Amrani, A., et al. 1996 J. Endocrinol. 148:139), IL-11 (Nicoletti, F., et al. 1999 Diabetes 48:2333), and IL-13 (Zaccone, P., et al. 1999 Diabetes 48:1522).

There remains a need in the art for other therapeutic regimens for the control of chronic inflammatory disorders, such as diabetes.

### BRIEF SUMMARY OF THE INVENTION

In one aspect, the invention provides a method of treating inflammation in a mammal comprising administering to the mammal an effective amount of allogeneic T lymphocytic cells. In one embodiment, the T cells are T-lymphoblastic leukemia (TALL) cells, or cells derived therefrom. In another embodiment, the T cells are xenogeneic. In one specific embodiment, the invention provides a method of treating inflammation in a mammal comprising administering to the mammal an effective amount of TALL-104 cells ATCC Accession No. CRL 11386. Preferably, the TALL-104 cells have been modified by stimulation in vitro by treatment with a cytokine and gamma irradiation at a dose suitable to irreversibly arrest cell proliferation, said modified cells characterized by irreversibly arrested cell proliferation and non-MHC restricted cytotoxic activity. In another specific embodiment, the invention provides a method of treating inflammation in a mammal comprising administering to the mammal an effective amount of the non-cytotoxic TALL-106 cells.

In another aspect, the invention provides a method of treating diabetes in a mammal comprising administering to the mammal an effective amount of allogeneic or xenogeneic T lymphocytic cells. In one embodiment, the T cells are T-lymphoblastic leukemia (TALL) cells, or cells derived therefrom. In a preferred embodiment, the T cells are TALL-104 cells, desirably modified as described above. In still another method, the T cells are TALL-106 cells. In this method the efficacy of the treatment is illustrated by a decrease in blood glucose levels.

Other aspects and advantages of the present invention are described further in the following detailed description of the preferred embodiments thereof.

### BRIEF DESCRIPTION OF THE FIGURES

FIG. 1A is a line graph illustrating glycemia measurement in five control female non-obese diabetic (NOD) mice, aged 5-6 weeks, that received 0.1 ml phosphate buffered saline (PBS) intraperitoneally (i.p.) every other week for 4 months (□) and in test group 1 female NOD mice (n=4) that received 1×10⁶ TALL-104 cells in 0.1 ml PBS, i.p., every other week from the 4-5^{th} week of age until sacrifice (⋄). The mean±SD for each group is shown on the graphs. Blood glucose levels were measured in each mouse every other week from the 12th week of age until sacrifice.

FIG. 1B is a line graph illustrating glycemia measurement in five control female non-obese diabetic (NOD) mice, aged 5-6 weeks, that received 0.1 ml phosphate buffered saline (PBS) intraperitoneally (i.p.) every other week for 4 months (□) and in test group 2 female NOD mice (n=4) that received 5×10⁶ TALL-104 cells in 0.1 ml PBS, i.p., every other week from the 4-5^{th} week of age until sacrifice (⋄).

The mean±SD for each group is shown on the graphs. Blood glucose levels were measured in each mouse every other week from the 12th week of age until sacrifice.

FIG. 1C is a line graph illustrating glycemia measurement in five control female non-obese diabetic (NOD) mice, aged 5-6 weeks, that received 0.1 ml phosphate buffered saline (PBS) intraperitoneally (i.p.) every other week for 4 months (□) and in test group 3 female NOD mice (n=4) that received 10⁷ TALL-104 cells in 0.1 ml PBS, i.p., every other week from the 4-5^{th} week of age until sacrifice (⋄). The mean±SD for each group is shown on the graphs. Blood glucose levels were measured in each mouse every other week from the 12th week of age until sacrifice.

FIG. 2 is a line graph illustrating glucose clearance, measured in mg/glucose/dl blood, after 4 months of treatment for the same mice, as in FIGS. 1A-1C, at two days before sacrifice, (i.e., at the 23rd week of age). Mice were deprived of food for 16 hours and the basal level of glucose was measured (mg glucose/dl blood at time 0). Mice were then injected with glucose (2 g/kg of body weight) into the tail vein. Blood glucose levels were measured again after 1, 2 and 4 hours in animals of group 1 (⋄) and controls (□), and after 1 and 2 hours in animals of groups 2 (◯) and 3 (Δ). Mean±SD for each group is shown.

FIG. 3 is a line graph illustrating glucose clearance after 3 months of treatment. Five- to 6-week-old female NOD mice were injected i.p. every other week with 0.1 ml PBS (□) or 2.5×10⁶ TALL-104 cells in 0.1 ml PBS (⋄) until sacrifice, for a total of 3 months. Two days before sacrifice (i.e., at 17-18th week of age), animals were deprived of food for 16 hours, the basal level of glucose was measured (mg glucose/dl blood at time 0), then glucose (2 g/kg of body weight) was injected into the tail vein. Blood glucose levels were measured again after 1 and 3.5 hours. Mean±SD for each group is shown.

FIG. 4 is a line graph illustrating the pancreatic accumulation index of ¹¹¹In-labeled TALL-104 cells in NOD (⋄) versus BALB/c (□) mice, aged 5-6 weeks. TALL-104 cells (2×10⁷ in 0.1 ml PBS) were injected into the tail vein of NOD and BALB/c mice. After sacrifice, blood volume was measured in a syringe, and the radioactivity of all organs and blood was counted in a γ-counter. Results are expressed as Relative Accumulation Index (RAI)=Cpm/mg tissue over CPM/μl blood.

FIG. 5 is a bar graph illustrating the distribution in pancreas, spleen, kidney and liver of ¹¹¹In-labeled TALL-104 cells in five groups of NOD mice, each indicated on the graph by a differently marked bar. ¹¹¹In-labeled TALL-104 cells (2×10⁷ in 0.1 ml PBS) were injected i.p. into NOD mice, aged 15 weeks. After 18 hours, mice were sacrificed, organs harvested, weighed, and radioactivity was measured in a γ-counter. Results are expressed as cpm/mg tissue×10⁷.

FIG. 6 is a line graph illustrating glycemia measurement in grams of glucose per dl/blood of female non-obese diabetic (NOD) mice, aged 5-6 weeks, at various hours after injection of 0.1 ml phosphate buffered saline (PBS) intraperitoneally (i.p.) (□), or 1×10⁶ TALL-104 cells in 0.1 ml PBS (◯) or 1×10⁶ TALL-106 cells in 0.1 ml PBS (⋄). Blood glucose levels were measured in each mouse at time 0, 1 hour, 2 hours and 3.5 hours post-injection.

### DETAILED DESCRIPTION OF THE INVENTION

The present invention is directed to a novel use of allogeneic T lymphocylic cells, which have been surprisingly found to have a role in the therapy of ongoing, chronic inflammatory processes and autoimmunity, particularly in diabetes, in mammals, particularly humans. However, it is anticipated that other mammals, e.g., veterinary patient, such as dogs, cats, horses, farm animals, etc., may be similarly treated. As used herein, the term “allogeneic T lymphocytes” means T lymphocytic cells that are antigenically distinct from T cells of the mammal receiving such cells. One subtype of allogeneic T cells for use in this invention are xenogeneic T cells. As used herein, the term “xenogeneic T lymphocytes” means having T lymphocytic cells that are from a different species than the mammal receiving the cells. Xenogeneic cells are, by definition antigenically distinct. Throughout this specification, as examples of particularly useful allogeneic T cells, among others, are modified TALL-104 cells and TALL-106 cells.

The Interleukin-2 (IL-2)-dependent MHC non-restricted cytotoxic T cell line derived from a patient with acute T lymphoblastic leukemia, TALL-104 (CD3+TCRα/β+ CDS⁺ CD16⁻) displays potent tumoricidal activity across species, sparing cells from normal tissues. The in vivo anti-cancer effects of TALL-104 cells have been documented both in experimental mouse models with implanted tumors and in canines with spontaneous malignancies has been successfully used in the treatment of experimental cancer in mice (Cesano, A. et al. 1994 J. Clin. Invest. 94:1076; Cesano, A. et al. 1995 Cancer Res. 55:96; Cesano. A. et al., 1996 Cancer Res. 56:4444; Visonneau, S. et al., 1997 Clin. Cancer Res. 3:1491; Cesano, A. et al. 1997 Anticancer Res. 17:1887; Cesano, A. et al., 1998 Anticancer Res. 18:2289; Geoerger, B. et al. 2000 NeuroOncology 2:103; and Kruse, C. A. et al. 2000 Cancer Res. 60:5731) and of naturally occurring malignancies in pet dogs and cats (Cesano, A. et al. 1996 Cancer Res. 56:3021; Visonneau, S., et al. 1997 Clin. Cancer Res. 3:1789; Visonneau, S., et al., 1999 Clin. Cancer Res. 5:1868; and Visonneau, S. et al. 1999 Oncol. Reports 6:1181). In tumor bearing mice, ¹¹¹Indium-oxiquinoline (¹¹¹In)-labeled TALL-104 cells were shown to infiltrate and accumulate preferentially at tumor site within 18 hours (Cesano, A. et al., 1999 Int. J. Oncol. 14:245).

Unmodified TALL-104 cells are available from the American Type Culture Collection, 10801 University Boulevard, Manassas, Va. 20110-2209 under Accession Number CRL 11386 and are described in detail in U.S. Pat. No. 5,272,082, incorporated herein by reference.

Other cells from childhood acute T-lymphoblastic leukemia include, among others, a cell line that was originally initiated in the presence of IL-3, but has now been determined to be growth factor independent, i.e., TALL-106 cells. See, e.g., R. O'Connor et al, 1991 Blood, 77(7):1534-45. These cells consists of fairly homogenous populations with a high nuclear cytoplasmic ratio and basophilic cytoplasms typical of L1 lymphoblasts, and express a T cell phenotype CD2⁺, CD7⁺, CD3⁺, CD8⁺, with the thymocyte marker, CD1a and TCRαβ⁺. TALL-106 fails to display cytotoxic activity under usual growth conditions or on induction with IL-2. TALL-106 cells were deposited with the American Type Culture Collection, 10801 University Boulevard, Manassas, Va. 20110-2209 on Feb. 15, 2002 and given Accession No. PTA-4085.

Both the TALL-104 and TALL-106 cells were deposited in compliance with the Budapest Treaty for the International Deposit of Microorganisms and were viable and capable of reproduction on the date of deposit. The deposits have been made under conditions of assurance of ready accessibility thereto by the public if a patent is granted. All restrictions imposed by the depositor on the availability to the public of the deposited material will be irrevocably removed upon the granting of a patent.

The novel therapeutic use of these allogeneic T lymphocytic cells, particularly TALL-104 and TALL-106 cells, is demonstrated in the examples below using an accepted animal model for chronic inflammation, which typically occurs in autoimmune disorders, i.e. the Non Obese Diabetic (NOD) mouse. The diabetic status in NOD mice is caused by a progressive infiltration of their pancreatic islets by autoreactive lymphocytes starting from 5-6 weeks of age that ultimately leads to the destruction of the β-cells and inability to produce insulin (Katz, J. D. et al. 1995 Science 268:1185). Mice become diabetic and die unless exogenous insulin is administered. In this strain of mice, over 90% of females develop age-related lymphocytic infiltration of the pancreatic islets which closely resembles the human type I diabetes (Atkinson, M. A. et al. 1999 Nature Med. 5:601). As the β-cells of the pancreatic islets are progressively destroyed by autoreactive lymphocytes, insulin is no longer produced. The animals develop diabetes around 5-6 months of age, and die of disease within two months.

As demonstrated in Examples 2 and 3 below, these mice were treated with TALL-104 cells starting from 5-6 weeks of age when infiltration of the pancreatic islets begins At this stage, the endothelial vessels of pancreatic β-islets express VCAM-1, MadCam-1, ICAM-1, ICAM-3, and CD31 (Hanninen, A. et al. 1993 J. Clin. Invest. 92:2509). By virtue of their high levels of expression of both the a4 and β1 chain components of the integrin α4β1 (ligand for the adhesion molecules expressed by the inflamed endothelia, VCAM-1) and of the β7 chain component of the integrin α4β7 (ligand for the endothelial adhesion molecule, MadCam) (Yang, X. D. et al. 1997 Diabetes 46:1542 and Michie, S. A. et al. 1998 Curr. Top. Microbiol. Immunol. 231:65), TALL-104 cells can adhere to the blood vessels of inflamed pancreatic islets.

As demonstrated in Example 4 below, upon administration of multiple injections over 3-4 months of modified, e.g., γ-irradiated (non-proliferating), cytokine-treated TALL-104 cells, NOD mice exhibited decreased levels of both lymphocytic infiltration of the pancreatic islets and glycemia, i.e., an improved ability to metabolize glucose as compared to untreated NOD mice. Migration studies using ¹¹¹Indium-oxiquinoline (¹¹¹In) labeled TALL-104 cells demonstrated a strikingly higher accumulation of these cells in the pancreatic islets of NOD mice, as compared to the other peritoneal organs and to the pancreas of healthy BALB/c mice.

As demonstrated by Example 5 below, TALL-104 cells were found to express high levels of adhesion molecules α4, α5, β1, β7 of the integrin family, and of ICAM-1, ICAM-3, LFA-1, LFA-3 of the Ig superfamily. This is consistent with their ability to thoroughly adhere to an inflamed tissue. Chemokine receptors CCR5, CXCR4, and CXCR3 were also found to be expressed on TALL-104 cells, indicating their potential to migrate toward inflamed tissues where chemokines, such as RANTES, macrophage inducible protein (MIP)-1α, and MIP-1β are produced. The observed protection of TALL-104 cells against pancreatitis and diabetes progression is due to the cytotoxic and/or cytostatic action of TALL-104 cells toward autoreactive host lymphocytes in the Langerhans islets, thus preventing islet destruction and allowing the production of normal insulin levels. Moreover, high expression of adhesion molecules of the Ig superfamily LFA-1, LFA-3, ICAM-I, and ICAM-3, which allows tight binding to inflamed endothelia, and of CD31, which is also involved in binding to the endothelia, supports the selective migratory ability of TALL-104 cells to inflamed tissues.

The infiltrating T cells in the pancreatic islets of NOD mice are mostly CD4⁺ Th1 lymphocytes (Pakala, S. V. M. et al. 1997 J. Exp. Med. 186:299). Chemokines produced by these cells include RANTES, MIP-1α, MIP-1β, and SDF (Baggiolini, M. et al. 1997 Annu. Rev. Immunol. 15:675). Indeed, these chemokines have been found in infiltrated islets of NOD mice (Bradley, L. M. et al. 1999 J. Immunol. 162:2511).

TALL-104 cells express mRNA for the CC chemokine receptors CCR1, CCR5, CCR2b, and the CXC chemokines CXCR4 and CXCR3. As demonstrated in the examples below, a similar pattern of chemokine receptors was found on LAK cells from healthy donors. Interestingly, TALL-104 express surface CCR5 (receptor for RANTES, MIP-1α, and MIP-1β), CXCR3 (receptor for Mig and IP-10), and CXCR4 (receptor for SDF-1) (Jung, S. et al. 1999 Curr. Opin. Immunol. 11:319), but lack CCR1 and CXCR5. Since TALL-104 cells express the receptors for the chemokines produced by inflamed pancreas, it is likely that they are chemotactically attracted there.

The adhesion molecules and chemokine receptors pattern found on TALL-104 cells supports the disclosure by the inventors that these cells selectively migrate to and accumulate in the pancreas of NOD mice as compared to the normal pancreas of healthy BALB/c mice. The data in these examples also showed that the level of pancreatitis in the TALL-104-treated animals was lower than in the PBS-injected group. In mice that received 107 cells per injection for 4 months, the majority of islets were free of lymphocytic infiltration. By contrast, age matched untreated animals showed high levels of infiltration.

While TALL-104 cells do not produce IL-4, they secrete abundant levels of IFN-γ, TNF-α, TNF-β, TGF-α, GM-CSF and IL-10 following stimulation with PMA, anti-CD3, and exposure to tumor cells. TALL-104 cells also express the message for IL-13 and its receptor, and constitutively produce low levels of IL-13 (results not shown). Thus, TALL-104 cells produce both pro-diabetic and anti-diabetogenic lymphokines. Because TALL-104 cells (CD3/TCRαβ+CDS+) do not react with mAb specific for Vα4 in inimunofluorescence assays (results not shown), they do not fall into the classical definition of NK T cells. However, similar to NK T cells, they are 90-100% CD161+ (results not shown), and are cytotoxic. It is presently theorized that, in the inflamed pancreas, TALL-104 cells release preferentially anti-inflammatory factors, resulting in the arrest of autoreactive lymphocyte recruitment and/or proliferation.

Following CD3 engagement or PMA treatment, TALL-104 cells release chemokines, including RANTES, MIP-1β, and MIP-1α, as measured by ELISA, and express the message for IP10 (not shown). It is theorized that after TALL-104 cells accumulate in the pancreatic islets, they secrete chemokines, which, in turn, contribute to the recruitment of host immune cells with anti-inflammatory properties (rather than β-cells reactive host lymphocytes) and consequently slow down the process of pancreatitis. The nature of the soluble factors released by TALL-104 cells and the identity of the host cells infiltrating the pancreatic islets of the NOD mice after administration of TALL-104 cells, are anticipated to underlie the protective effect of TALL-104 cell treatment.

The protective effect of TALL-104 cells may also result because they lyse the autoreactive infiltrating lymphocytes in the pancreatic islets. While preliminary experiments testing the ability of TALL-104 cells to kill splenic lymphocytes of NOD mice in vitro do not appear to support this hypothesis, TALL-104 cells might only lyse activated autoreactive T cells which express an antigen recognized by TALL-104 cells. Because this T cell subset represents only a low percent of the total splenic population, a cytotoxic action of TALL-104 cells toward infiltrating lymphocytes can be missed in an in vitro assay using bulk splenocytes as target.

In conclusion, the data presented below in the examples indicate that TALL-104 cells, by virtue of their adhesive molecules and chemokine receptor pattern, are able to migrate and accumulate in the inflamed pancreatic islets of pre-diabetic NOD mice. By secreting anti-inflammatory factors and/or by directly killing autoreactive lymphocytes, TALL-104 cells accomplish an anti-inflammatory action which slows down the inflammatory process and, in turn, preserves the pancreatic β-cells. As a result, mice treated with TALL-104 cells maintain their ability to produce insulin and are thereby protected from diabetes. This protection may be a direct effect of repeated exposure to TALL-104 cells or may occur indirectly through the activation of the host's immune system. A study of the ability of NOD mice to mount xenogeneic immune responses indicates that antibodies against TALL-104 cells are present in the circulation after six TALL-104 cell injections. TALL-104 cells are, therefore, able to activate host cells, triggering a series of reactions which may lead to an anti-inflammatory effect. If halting TALL-104 cell therapy results in the reappearance of the diabetic condition in the responsive mice, periodic TALL-104 cell administrations to keep the disease under control may be necessary.

Further, as demonstrated in FIG. 6 and discussed in Example 2D, the effect of the TALL-104 cells on untreated diabetic mice (controls) was compared with the effect of non-cytotoxic TALL cells, e.g., TALL-106 cells, on untreated diabetic mice. The mice's response to the non-cytotoxic T lymphocytic TALL-106 cells was better than the reaction to the modified, cytotoxic TALL-104 cells. Therefore, it is anticipated that a suitable dosage of non-cytotoxic T lymphocytic cells may also be employed in this treatment protocol. There are a number of non-cytotoxic TALL cells, such as those described in R. O'Connor, cited above and incorporated by reference herein. These TALL cells are intended to be encompassed in this invention.

Given this data, therefore, the present invention involves a method of treating chronic inflammatory conditions in a mammal. More preferably, the treated mammal is a human. Among such chronic inflammatory conditions are included, without limitation, diabetes, schleroderma, asthma, autoimmune hemolytic anemia, pernicious anemia, thyroiditis, chronic lymphocytic thyroiditis, allergic encephalomyelitis, nephritis, Multiple Sclerosis, Graves' disease, myasthenia gravis, pemphigus, Crohn's disease, systemic lupus erythematosus, and rheumatoid arthritis. For clarity of discussion, the method of this invention is discussed below with relation to the chronic inflammatory condition of diabetes. However, one of skill in the art can readily adapt these methods to the treatment of other chronic inflammatory disorders.

The method involves administering to the mammal an effective amount of suitable allogeneic T lymphocytic cells. As one example, the T cells include TALL-104 cells which have been modified by stimulation ill vitro by treatment with a cytokine and gamma irradiation at a dose suitable to irreversibly arrest cell proliferation. These modified TALL-104 are cells characterized by irreversibly arrested cell proliferation and non-MHC restricted cytotoxic activity. As discussed above, the method of the invention utilizes modified TALL-104 cells, which are derived from unmodified TALL-104 cells, ATCC Accession Number CRL 11386.

TALL-104 cells may be modified in such a way as to provide them with an increased cytotoxicity but arrested proliferation. Such modification methods have been described in detail in International Patent Publication No. WO94/26284, published Nov. 24, 1994, and in U.S. Pat. No. 5,683,690, which are incorporated by reference herein.

For example, one modification step includes in vitro treatment of the TV 104 cells with a selected cytokine or combination of cytokines. Among suitable cytokines for the modification of TALL-104 cells are the interleukins, particularly interleukin-2, interleukin-12 and interleukin-15. Preferably for this modification, the interleukins are recombinant human (rh) lymphokines. These interleukins, when used independently to treat the cell line, induce the cell line's cytotoxic activity. When these cytokines are used together to modify the cell line, the modified cell line displays additive or increased cytotoxic effects. This results in a significant increase in cytotoxic activity and recycling capability (Cesano et al, J. Immunol., 151:2943 (1993)).

Another modification step involves the exposure of the TALL-104 cell line to lethal irradiation to confer irreversible loss of growth capability with full retention of cytotoxic activity, both in vitro and in vivo. This is achieved by subjecting the cell lines to y-irradiation just prior to their use. Preferably, the cells are irradiated at 4000 rads using a ¹³⁷CS source. As described in International Patent Publication No.

WO94/26284, irradiation of TALL-104 cells provides a modified cytotoxic cell line that has lost its proliferative ability and, therefore, the possibility of growing in an unrestrained fashion in the recipient organism. In fact, unlike their non-irradiated counterparts, modified γ-irradiated TALL-104 cells of this invention transplanted into SCID mice do not cause leukemia.

In one embodiment, modified TALL-104 cells are prepared as follows. TALL-104 cells (ATCC CRL 11386) are exponentially grown in tissue culture in the presence of recombinant human (rh) IL 2. If desired, IL-12 can be added for about 18 hours before use to enhance the killing activity of the cell line. The cytokine-treated TALL-104 cells are then γ-irradiated, preferably at about 4,000 rads. The resulting cell line is referred to as the modified TALL-104 cell line.

As another example, the T cells include TALL-106 cells which are exponentially grown in tissue culture. The growth factor-independent TALL-106 cells are preferably treated with gamma irradiation at a dose suitable to irreversibly arrest cell proliferation, e.g., at about 4,000 rads, to arrest unwanted cell proliferation before administration.

Desirably in the practice of this invention, the allogeneic T lymphocytic T cells (e.g., TALL-104 or TALL-106 cells) are administered in a pharmaceutically acceptable carrier. One particularly desirable carrier is saline or buffered saline. Another desirable carrier is plasma protein fraction 5% with 10% dimethylsulfoxide (DMSO). However, other suitable carriers are well known to those of skill in the art. The selection of the carrier is not a limitation on the present invention.

Additionally, the allogeneic T lymphocytic cells may be administered to the patient with other therapeutic agents suitable for combined treatment of the particular chronic inflammatory condition. Such additional therapeutic agents include non-steroidal anti-inflammatory drugs, corticosteroids, Cos-2 inhibitors, cytotoxic agents, such as methotrexate, anti-hyperglycemic agents, preferably oral anti-hyperglycemic agents (e.g., insulin, sulfonyl urea and metformin), antibodies to cytokines and antibodies to tumor necrosis factor (TNF) receptors, among others.

Other components, such as inert components, may be added to tie allogeneic T lymphocytic cell-containing composition to assist in a particular mode of delivery. Some specific examples include, without limitation, a topical solution, a creme or ointment for application to the skin, a solution or ointment suitable for application to the eye, a solution or spray suitable for application to the throat, and a solution suitable for application to the gums. Formulation of such delivery compositions containing the allogeneic T lymphocytic cells (e.g., TALL-104 or TALL-106 cells) is within the skill of the art.

The amount of the allogeneic T lymphocytic cells (e.g., TALL-104 or TALL-106 cells) present in each effective dose is selected with regard to a variety of considerations. Among such considerations are the type, identity and severity of the inflammatory response and its cause, the location of the inflammatory response (e.g., systemic or localized), the type of mammal, the mammalian patient's age, weight, sex, general physical condition and the like. The amount of allogeneic T lymphocytic cells required to induce a therapeutic effect without significant adverse side effects varies depending upon the other pharmaceutical or veterinary agents employed in the therapy of the patient and the optional presence of other components or therapeutic reagents and the like, in the allogeneic T lymphocytic cell-containing composition itself.

Generally, each dose contains between about 106 to about 10⁸ allogeneic T lymphocytic cells (e.g., modified TALL-104 and/or TALL-106 cells)/kg patient body weight suspended in a suitable amount of a pharmaceutically acceptable carrier. A more preferred dosage is about between 10⁷ to about 10⁸ allogeneic T lymphocytic cells/kg patient body weight. Other dosage ranges are contemplated by one of skill in the art. This dose may be formulated in a pharmaceutical composition, as described above (e.g., suspended in about 0.01 mL to about 1 mL of a physiologically compatible carrier) and delivered by any suitable means.

For the treatment of chronic inflammatory disorders, e.g., diabetes, the initial dosage of the allogeneic T lymphocytic cells are desirably followed by repeated administration for a duration selected by the attending physician. Dosage frequency depends upon the factors identified above. As one example, dosage ranges from 1 to 6 doses per day for a duration of about 3 days to a maximum of no more than about 1 week. Alternatively, allogeneic T lymphocytic cells may be administered daily for five consecutive days, and a booster administered after approximately one month. In another example, the allogeneic T lymphocytic cells are administered every other day over the course of two weeks, with cell boosts administered monthly for a desired period of time, e.g., six months. Such boosts may be administered for one to two consecutive days at the doses set forth above. Suitable dosages may be delivered daily, weekly, bi-weekly, monthly or at any suitable interval. Alternatively, for chronic inflammatory conditions, dosage protocols will involve long-term administration of the allogeneic T lymphocytic cells. Suitable dosage protocols are selected by the attending physician. The timing, dosage and course of administration and determination as to the number and timing of boosters desired may be readily determined by one of skill in the art. The timing and the mode of administration do not limit the present invention.

In the practice of this method of the invention, the allogeneic T lymphocytic cells may be administered by any routes of administration that is suitable to treat the particular inflammatory condition. Such routes include, without limitation, intravenous, intradermal, transdermal, intraperitoneal, intramuscular, intrathecal, subcutaneous, mucosal, and intraocular. A preferred route may be by injection, e.g., intravenously or intramuscularly. Where desired or necessary, the allogeneic T lymphocytic cells may be administered locally, i.e., to the site of localized inflammation, or to a specific organ. For example, the allogeneic T lymphocytic cell may be delivered via a shunt to an organ, e.g., the pancreas, for the treatment of diabetes. Generally, where the allogeneic T lymphocytic cells are administered locally, the amount of cells administered in a single or repeated dosage is considerably lower than the range provided above. For example, local administration of only 10⁹ cells in total over the course of a week.

The following examples, including those discussed briefly above, illustrate several embodiments of this invention, particularly the treatment of diabetes in a diabetic mouse model with TALL-104 or TALL-106 cells, as a suitable example of a chronic inflammatory condition. These examples are illustrative only, and do not limit the scope of the present invention.

### EXAMPLE 1

### Preparation of Cells

TALL-104 cells were cultured in Iscove's modified Dulbecco's medium (IMDM, Gibco-BPL, Grand Island, N.Y.) supplemented with 10% heat inactivated fetal bovine serum (FBS; Atlanta Biological, Norcross, Ga.), and 100 U/ml recombinant human (rh)IL-2 (Chiron, Emeryville, Calif.), in a humidified 10% CO₂ incubator. For radiolabelling, TALL-104 cells were suspended at 10⁸/ml in IMDM in the presence of 200 μCi ¹¹¹Indium-oxyquinoline (Amersham International, Arlington Heights, Ill.) for 30 minutes at 37° C., washed three times, and re-suspended in PBS at 2×10⁸/ml.

TALL-106 cells were cultured in Iscove's modified Dulbecco's medium (IMDM, Gibco-BPL, Grand Island, N.Y.) supplemented with 10% heat inactivated fetal bovine serum (FBS; Atlanta Biological, Norcross, Ga.), in a humidified 10% CO₂ incubator. For radiolabelling, TALL-106 cells were suspended at 10⁸/ml in IMDM in the presence of 200 μCi ¹¹¹Indium-oxyquinoline (Amersham International, Arlington Heights, Ill.) for 30 minutes at 37° C., washed three times, and re-suspended in PBS at 2×10⁸/ml.

Peripheral blood mononuclear cells (PBMC) from healthy human donors were isolated by Ficoll gradient centrifugation (Gibco-BRL) and either used fresh or frozen until needed. These cells are used in the following experiments for injection into mice to ascertain the effect of normal, xenogeneic T lymphocytes on a mammal.

Lymphokine activated killer (LAK) cells were generated by culturing monocyte-depleted PBMC in the presence of 100 U/ml rhIL-2 for 5-15 days. Depletion of CD4+ T cells was achieved using magnetic beads (Dynal, Lake Success, N.Y.), according to the manufacturer's instructions.

### EXAMPLE 2

### Effect of TALL-104 Cell Administration on Blood Glucose Level and Glucose Clearance

A. The following experiment demonstrates the effect of TALL-104 cell administration on non-obese diabetic (NOD) mice and evaluates the influence of this treatment on the development of diabetic status. Glycemia levels were compared in TALL 104 cell-treated versus untreated NOD mice.

Animals, female NOD and BALB/c mice at 5-6 weeks old (Taconic Laboratories, Bar Harbor. ME) were divided into 4 groups. The control group (n=5) was injected i.p. with 0.1 ml PBS every other week for 4 months, while the 3 experimental groups (n=4/group) received i.p. injections of γ-irradiated (40 Gy), non-proliferating, TALL-104 cells at doses of 106 cells (group 1), 5×10⁶ cells (group 2), or 10⁷ cells (group 3), respectively, in 0.1 ml PBS every other week until sacrifice, for a total of 4 months.

Animals were checked every other week for glucose levels starting from the 12th week of age by collecting blood samples from either the retroorbital venous plexus or the tail vein of NOD mice. The blood glucose levels were measured using Accu-Chek strips (Roche, Indianapolis, Ind.).

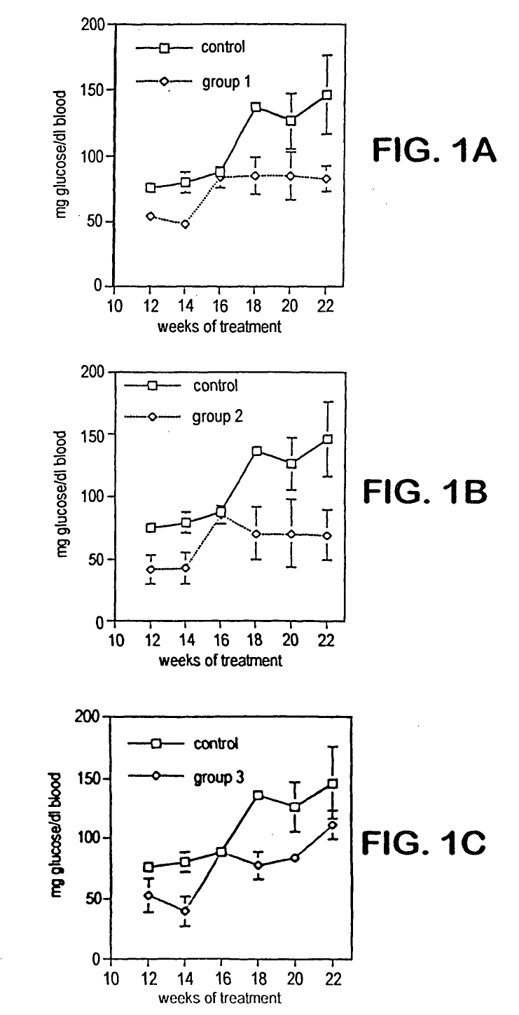

The results are illustrated in FIGS. 1A, 1B and 1C. Of the five control animals, two (mouse #2 and 5) became hyperglycemic (glucose level >250 mg/dl) and died in the 16th and 20th week of age, respectively. The 3 surviving control animals showed glucose levels of 150 mg/dl in the 18th week of age which then plateaued or increased later on. By contrast, in all 3 groups of TALL-104 treated animals, the level of blood glucose never exceeded 100 mg/dl throughout the 4-month treatment. Interestingly, in group 3 mice treated with the highest TALL 104 cell concentration (FIG. 1C), an increase in glycemia was noted in the 22nd week.

B. Before reaching a high blood glucose level, animals with a compromised ability to produce insulin also display an impaired glucose clearance from the circulation. Glucose clearance was therefore, measured on the same animals 2 days before sacrifice.

Four- to five-week-old female NOD mice were divided into the same 4 groups as described above. Control mice were injected i.p. with 0.1 ml PBS. Groups 1, 2, and 3 mice were injected with 0.1 ml γ-irradiated (40 Gy) TALL-104 cells containing 1×10⁶ cells, 5×10⁶ cells, or 10⁷ cells in PBS, respectively. Each injection was repeated every other week, until sacrifice, for a total of 4 months. Glucose level was measured every other week using AccuChek strips, as described above.

To test for the ability to clear glucose from the blood, 2 days before sacrifice, the mice were deprived of food for 16 hours and the basal glucose level was measured. Mice were then injected i.v. into the tail vein with 2 kg of body weight of a glucose solution in PBS. Blood samples were collected 1, 2 and 4 hours later for glycemia values, i.e., glucose determinations.

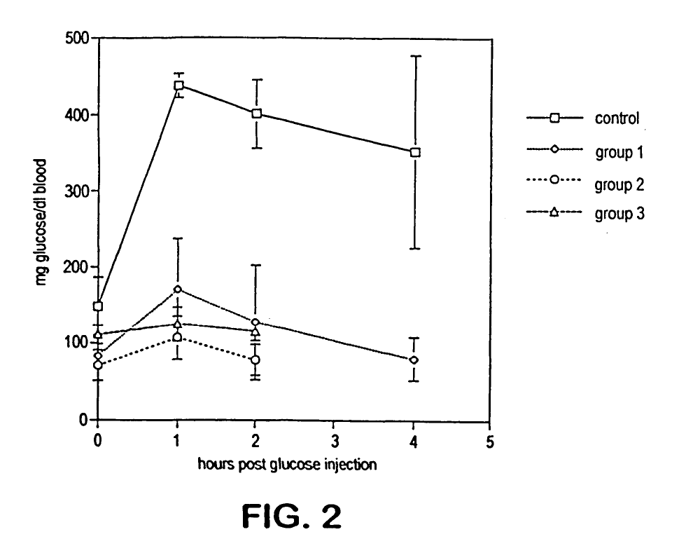

Results, expressed as mg of glucose per dl of blood for each time point (mean±S.D.) are illustrated in FIG. 2. At 4 hours, the 3 surviving control mice still had mean glycemia values of 200 mg/ml over the basal level. By contrast, in groups 2 and 3 that received 5×10⁶ and 10×10⁶ TALL-104 cells, respectively, the mean glucose level was back to the basal level within 1 hour. The mean glucose level for group 1 mice that received 1×10⁶ TALL-104 cells was down to the basal level within 2-4 hours.

C. Another experiment was performed to investigate the effect of a shorter treatment with TALL-104 cells on the progression of diabetes. Sixteen female NOD mice, aged 5-6 weeks, were divided into two groups. Control mice (n=8) received 0.1 ml PBS, and the experimental group (n=8) was injected with 2.5×10^{6 TALL-}104 cells in 0.1 ml PBS. Both experimental and control mice were injected i.p. every other week for a total of 3 months, and then sacrificed. Two days before sacrifice, animals were deprived of food for 16 hours. The blood glucose level was measured (basal level), by the procedures described above. Immediately afterwards, 2 g of glucose per kg of body weight was injected into the tail vein. glucose was measured again after 1 and 3.5 hours.

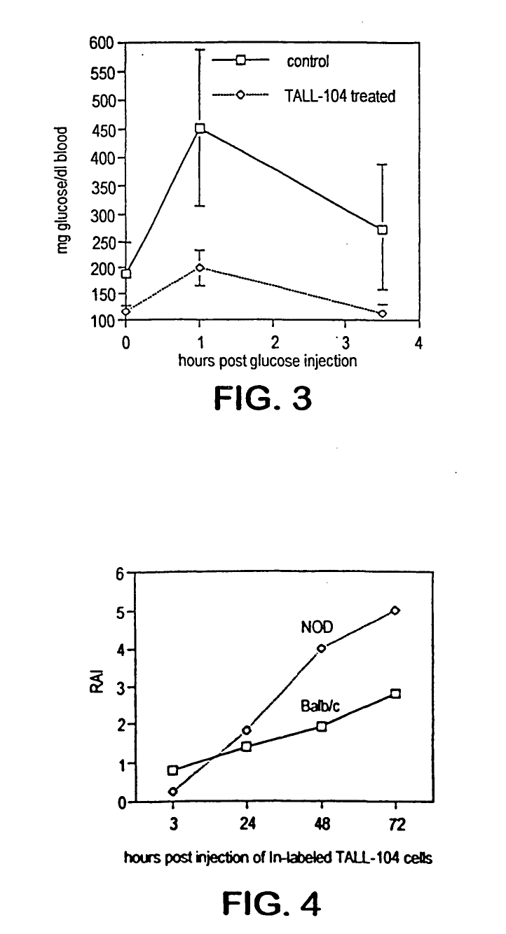

Results were expressed as mg of glucose per dl of blood (mean±S.D.) for each time point and illustrated in FIG. 3. In this experiment, two control animals became diabetic and died at age 12 and 14 weeks, respectively. For the remaining mice in the control group, the mean glycemia level, 1 hour after glucose administration, was 250 mg glucose per dl blood higher than the basal level. By contrast, in the TALL-104 cell-treated mice, 1 hour after glucose injection, the mean glycemia level exceeded the basal level only by 100 mg glucose per dl blood.

D. Another experiment compares the effect of TALL-104 vs. non-cytotoxic TALL-106 cell administration on non-obese diabetic (NOD) mice and evaluates the influence of this treatment on the development of diabetic status for several hours post-injection. Glycemia levels were compared in TALL-104 cell-treated versus TALL, 106 cell-treated, versus untreated NOD mice.

Animals, female NOD and BALB/c mice at 5-6 weeks old (Taconic Laboratories, Bar Harbor, Me.) were divided into 4 groups. The control group (n=5) was injected i.p. with 0.1 ml PBS, while the 2 experimental groups (n=4/group) received i.p. injections of either γ-irradiated (40 Gy), non-proliferating, TALL-104 cells at doses of 10⁶ cells in 0.1 ml PBS or y-irradiated (40 Gy) TALL-106 cells at doses of 10⁶ cells in 0.1 ml PBS Animals were checked periodically for up to about 4 hours post-injection for glucose levels by collecting blood samples from either the retroorbital venous plexus or the tail vein of NOD mice. The blood glucose levels were measured using Accu-Chek strips (Roche, Indianapolis, Ind.).

The results are illustrated in FIG. 6 and expressed as g of glucose per dl of blood (mean±S.D.) for each time point. For the first 2 hours post-injection, the animals treated with non-cytotoxic, allogeneic T lymphocytic cells, TALL-106 maintained consistently low levels of glucose (under about 130 g) in contrast to the high glucose levels (as high as 250 g) revealed by the control untreated animals. The results with TALL-106 cells were better than those animals treated with TALL-104 cells for the first 2 hours post-injection.

In an analogous experimental protocol, the normal human PBMCs at a similar dosage are injected into the NOD mice according to a similar protocol to determine the effect of normal xenogeneic T cells on the mice.

### EXAMPLE 3

### The Level of Pancreatitis in TALL-104 Treated NOD Mice

The inability to metabolize glucose, which ultimately leads to the diabetic status, is due to the destruction of β-cells in the pancreatic Langerhans islets by auto-reactive lymphocytes. To ascertain whether the observed improvement in metabolizing glucose (e.g., measuring circulating or blood glucose) in the TALL-104-treated animals in Example 2 correlated with a lower degree of pancreatic islets infiltration, the following experiments were performed.

Pancreata from cell-treated and untreated NOD mice of Example 2 were frozen in OCT. Five gm sections were obtained in a cryostat, fixed in acetone (Fisher, Pittsburgh, Pa.), and stained with hemathoxylin-cosin (H&E). After treatment with ethanol and xylene (Fisher), slides were analyzed by light microscopy for the presence and extent of infiltrating lymphocytes.

The results of this experiment are illustrated in Table 1, in which a score for the level of lymphocytic infiltration in each islet was assigned as follow: −, no infiltration; +, peninsular infiltration; ++, up 50% of the islet was infiltrated; +++, more than 50% of the islet was infiltrated. The % islets that scored −, +, ++, or +++was calculated for each group of animals. Differences between the experimental and control animals (mean±SD) were analyzed for statistical significance using the Studcnt's t test for paired data.

Micrographs of the pancreatic islets of TALL-14 treated and PBS-injected control animals (not shown) illustrated reduced lymphocytic infiltration of pancreatic islets in animals treated with TALL-104 cells. Many pancreatic islets from TALL-104-treated animals were either modestly infiltrated or free from infiltration, while all islets from the untreated mice were heavily infiltrated or, in some cases, destroyed. Results were similar in mice that were treated for 4 or 3 months.

TABLE 1
Infiltration Score of Pancreatic Islets

| | | % islets with | % islets with 50% |
|---|---|---|---|
| | Length of | no (−) or | (++) of >50% |
| | treatment | periinsular (+) | (+++) |
| Mice treated with | (mos) | infiltration | infiltration |
| PBS | 4 | 0 | 100 |
| TALL-104 (1 × 10⁶) | 4 | 50* | 50* |
| TALL-104 (5 × 10⁶) | 4 | 75* | 25* |
| TALL-104 (10 × 10⁶) | 4 | 93* | 7* |
| PBS | 3 | 67 | 33 |
| TALL-104 (2 × 10⁶) | 3 | 95* | 5* |

*p < 0.005 for each group of TALL-104-treated mice vs. control (PBS-injected) mice by Student's t test.

These data, taken with the data from Example 2, indicate that administration of TALL-104 cells improves the ability of NOD mice to metabolize glucose most likely by preventing pancreatic 1-cell destruction.

### EXAMPLE 4

### Distribution of TALL-104 Cells in NOD vs BALB/c Mice

In order to test whether TALL-104 cells preferentially home to inflamed pancreatic islets, ¹¹¹In-labeled TALL-104 cells were injected in diseased NOD mice and healthy BALB/c mice, and their accumulation in the pancreas of both groups of mice was compared.

A. Two×10^{7 111}In-labeled TALL-104 cells in 0.1 ml PBS were administered i.v. into the tail vein of 5 to 6-week-old NOD and BALB/c mice. At this age, a low peri-insular infiltration is usually observed. Animals were sacrificed after 3, 24, 48, or 72 hours, their pancreata were harvested, weighed, and radioactivity was measured in a y-counter. Cpm/mg of tissue were normalized for the ¹¹¹In-oxine decay (half life of 3 days) by dividing for cpm/μl blood. The relative accumulation index (RAI) was calculated according to the formula:

RAI=(Cpm/mg tissue)/(Cpm/μl blood).

Results are displayed graphically in FIG. 4, which indicated that TALL-104 cells accumulated in the pancreas of NOD mice over time, with an accumulation index higher than that observed in healthy BALB/c mice.

B. To determine whether tissue inflammation correlated with TALL-104 cell homing to the inflammatory site, 2×10^{7 111}In-labeled TALL-104 cells in 0.1 ml PBS were injected i.p. into female 15-week old NOD mice. The majority of animals at this age had a high level of pancreatic lymphocyte infiltration. After 18 hours, pancreas, spleen, kidney, and liver, were harvested, weighed, and radioactivity was measured (expressed as cpm/mg tissue). For histological examination, the pancreata used for the evaluation of TALL-104 accumulation after i.p. injection were frozen in OCT, cut in a cryostat, and stained with H&E, as described above.

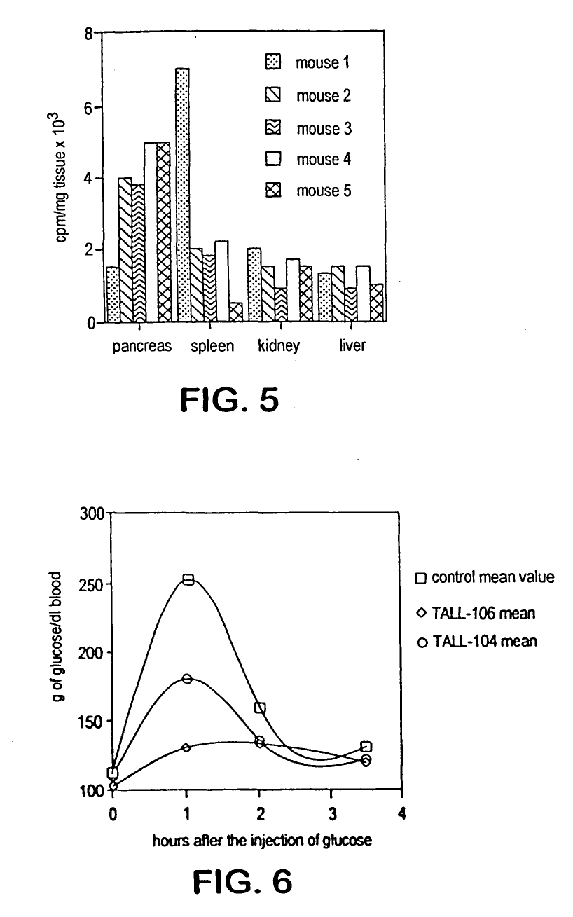

The results of the radioactivity are illustrated in the graph of FIG. 5, which shows that in 4 out of 5 NOD mice, TALL-104 cells were found predominantly in the pancreas as compared to the other organs. In the histological sections (not shown), H&E staining showed lower lymphocytic infiltration in the pancreatic islets of the mouse in which no accumulation of radiolabled TALL-104 cells was found, as compared to the pancreata of the other 4 mice which contained high numbers of radiolabeled TALL-104 cells.

Taken together, these results indicate that TALL-104 cells have a high tendency to migrate to inflamed pancreata.

### EXAMPLE 5

### Adhesion Molecules and Chemokine Receptors Expression by TALL-104 Cells

For lymphocytes to extravasate and reach an inflamed parenchyma, they need to express a particular pattern of adhesion molecules and chemokine receptors. Adhesion molecules will provide tight adhesion to the endothelia by binding to the endothelial ligands, while chemokine receptors will allow the cells to follow a chemotactic gradient toward the inflamed parenchyma. This phenotypic profile is typical of activated-memory T cells, as naive T cells are unable to infiltrate inflamed tissues. To test whether TALL-104 cells express the adhesion molecules and chemokine receptors required for migrating to and homing in an inflamed parenchyma, the following procedures were performed.

TALL-104 cells, cultured for 8 weeks in rhIL-2, were analyzed for surface expression of adhesion molecules and chemokine receptors. Analysis was performed by flow cytometry as described below using antibodies specific to human CD2 (RPA-2.10), CD11/LFA-1 (HI111), CD29/β1 (MAR4), CD31 (WM59), CD44 (G44-26), CD49d/α4 (9F10), CD54/ICAM-1 (HA58), CD50/LFA-3 (TU41), CD62L (SH-11) CCR5 (2D7/CCR5), and CXCR4 (12G5), all purchased from Pharmingen, San Diego, Calif. Antibodies to CXCR1 (42705.111), CXCR2-(48311.211), CXCR3 (49801.111), CXCR5 (51505.111), and CCR1 (53504.111) were purchased from R&D Systems, Minneapolis, Minn. A fluoresccin-conjugated secondary antibody against mouse IgG F(ab)₂ fragment was also employed (Organon Teknika Corporation, Durham, N.C.).

A. RNA Extraction

mRNA was extracted from 2×10⁷ TALL-104 cells or 2×10⁷ CD4depleted LAK cells from a healthy volunteer. RNA was extracted from TALL-104 and LAK cells with RNA Stat-60 (Tcl-Test, Inc., Friendswood, Tex.) according to the manufacturer's instructions. Isopropanol, chloroform, and ethanol were purchased from Fisher, ultrapure water was obtained by adding 0.5% diethylpyrocarbonate (Sigma, St Louis, Mo.) to bidistilled water and autoclaved for 30 minutes. Messages for various chemokine receptors were detected by RPA, as described below.

B. RNAse Protection Assay (RPA)

Detection of mRNA for chemokine receptors was performed by RiboQuant MultiProbe RNAse Protection Assay System (Pharmingen), according to the manufacturer's instructions. Briefly, the DNA template was used to synthesize the ³²P-labeled anti-sense RNA probe. The probe was hybridized in excess of sample RNA. Free probe and single-stranded RNA were digested with RNAse. The remaining RNAse protected-probes were resolved on polyacrylamide gels, and quantified by phosphoimaging. Protected RNA of the chemokine receptors was identified by comparing their electrophoretical mobility with that of standard RNA. The RNA for the housekeeping genes L32 and GAPDH were included in the template set as quality control, a pattern similar to that of CD4-depleted human LAK cells.

C. Results

By immunofluorescence analysis, TALL-104 cells were found to express high levels (=90%) of the integrin chains, α4, α5, α1, α7, components of the integrins a4β1, α5β1, and α4β7, respectively. Most of the cells (=90%) expressed surface CD44 and CD31. Adhesion molecules of the Ig superfamily LFA1, LFA3, ICAM-1, ICAM-3, CD44 and CD31, were found on 60-100% of TALL-104 cells. By contrast, TALL-104 cells have low levels of L-selectin, the adhesion molecule involved in migration to lymph nodes but not to inflamed areas. Immunofluorescence analysis confirmed the expression of surface CXCR3, CXCR4, and CCR5 receptors on TALL-104 cells; however, CCR1 was not detectable. Overall this phenotypic profile of TALL-104 cells is indicative of their ability to adhere to the endothelia of an inflamed tissue.

After achieving tight adhesion and extravasation, cells must be able to respond to chemotactic stimuli secreted by the inflamed tissues in order to penetrate the parenchyma. By the RNAse Protection Assay, TALL-104 cells were found to express mRNA for CCR5, CCR1, CCR8, CCR2b, CXCR3 and CXCR4.

These data indicate that TALL-104 cells could be attracted by several chemokines, including RANTES, macrophage inducible protein (MIP)-1β, MIP-1βstromal cell-derived factor (SDF), and monokine induced by interferon-α (Mig). Taken together, the pattern of adhesion molecules and chemokine receptors on TALL-104 cells supports their ability to extravasate and home to an inflamed tissue.

Numerous modifications mid variations of the present invention, are included in the above-identified specification and are expected to be obvious to one of skill in the art. Such modifications and alterations to the methods of the present invention are believed to be encompassed in the scope of the claims appended hereto.

1. A method of treating diabetes in a mammal comprising administering to the mammal an effective amount of allogeneic lymphocytic T cells.

2. The method according to claim 1, wherein said T cells are xenogeneic cells.

3. The method according to claim 1, wherein said T cells are TALL-104 cells ATCC Accession No. CRL 11386, which cells have been modified by stimulation in vitro by treatment with a cytokine and gamma irradiation at a dose suitable to irreversibly arrest cell proliferation, said modified cells characterized by irreversibly arrested cell proliferation and non-MHC restricted cytotoxic activity.

4. The method according to claim 3, wherein the cytokine is selected from the group consisting of IL-2, IL12 and IL15.

5. The method according to claim 1, wherein said T cells are TALL-106 cells ATCC Accession No. ______.

6. The method according to claim 5, wherein said TALL-106 cells are gamma-irradiated at a dose suitable to irreversibly arrest cell proliferation.

7. The method according to claim 1, wherein the mammal is a veterinary patient.

8. The method according to claim 1, wherein the mammal is a human.

9. The method according to claim 1, wherein the effective amount is between 10⁶ to about 10⁸ cells/kg.

10. The method according to claim 1, wherein the cells are administered by a route selected from the group consisting of intramuscular, intravenous, intradermal, transdermal, intraperitoneal, intrathecal, subcutaneous, mucosal, and intraocular.

11. The method according to claim 1, wherein the cells are administered via direct shunt.

12. The method according to claim 1, wherein the allogeneic T cells are administered to said mammal daily for up to two weeks.

13. The method according to claim 1, wherein the allogeneic T cells are administered to said mammal on a schedule selected from the group consisting of daily, weekly, bi-weekly and monthly.

14. The method according to claim 1, wherein said method further comprises the step of co-administering, to said mammal a therapeutic agent selected from the group consisting of non-steroidal anti-inflammatory drugs, corticosteroids, Cos-2 inhibitors, cytotoxic agents, anti-hyperglycemic agents, insulin, sulfonyl urea, metformin, antibodies to cytokines and antibodies to tumor necrosis factor receptors.

15. The method according to claim 1, wherein said administration decreases circulating glucose levels in said mammal.

16. A method of treating inflammation in a mammal comprising administering to the mammal an effective antiinflammatory amount of allogeneic lymphocytic T cells.

17. The method according to claim 16, wherein said T cells are xenogeneic cells.

18. The method according to claim 16, wherein said T cells are TALL-104 cells ATCC Accession No. CRL 11386, which cells have been modified by stimulation in vitro by treatment with a cytokine and gamma irradiation at a dose suitable to irreversibly arrest cell proliferation, said modified cells characterized by irreversibly arrested cell proliferation and non-MHC restricted cytotoxic activity.

19. The method according to claim 18, wherein the cytokine is selected from the group consisting of IL-2, IL12 and IL-15.

20. The method according to claim 16, wherein said T cells are TALL-106 cells ATCC Accession No. ______.

21. The method according to claim 17, wherein said TALL-106 cells are gamma-irradiated at a dose suitable to irreversibly arrest proliferation.

22. The method according to claim 16, wherein the mammal is a veterinary patient.

23. The method according to claim 16, wherein the mammal is a human.

24. The method according to claim 16, wherein the effective amount is between 10⁶ to about 10⁸ cells/kg.

25. The method according to claim 16, wherein the cells are administered by a route selected from the group consisting of intramuscular, intravenous, intradermal, transdermal, intraperitoneal, intrathecal, subcutaneous, mucosal, and intraocular.

26. The method according to claim 16, wherein the cells are administered via direct shunt to the site of inflammation.

27. The method according to claim 16, wherein the allogeneic T cells are administered to said mammal on a schedule selected from the group consisting of daily, weekly, bi-weekly and monthly.

28. The method according to claim 16, wherein said method further comprises the step of co-administering to said mammal a therapeutic agent selected from the group consisting of non-steroidal anti-inflammatory drugs, corticosteroids, Cos-2 inhibitors, cytotoxic agents, anti-hyperglycemic agents, insulin, sulfonyl urea, metformin, antibodies to cytokines and antibodies to tumor necrosis factor receptors.

29. Use of allogeneic lymphocytic T cells in the preparation of a medicament for the treatment of diabetes in a mammal.

30. Use according to claim 29, wherein said cells are TALL-104 or TALL-106 cells.

31. Use of allogeneic lymphocytic T cells in the preparation of a medicament for the treatment of inflammation in a mammal.

32. Use according to claim 31, wherein said cells are TALL-104 or TALL-106 cells.
